(19) United States
(12) Patent Application Publication
(10) Pub. No.: US 2025/0143586 A1
(43) Pub. Date: May 8, 2025

(54) SYSTEM AND METHOD FOR EVALUATING CARDIAC PUMPING FUNCTION

(71) Applicant: Guy P. Curtis, San Diego, CA (US)

(72) Inventor: Guy P. Curtis, San Diego, CA (US)

(21) Appl. No.: 19/017,712

(22) Filed: Jan. 12, 2025

Related U.S. Application Data
(62) Division of application No. 17/580,300, filed on Jan. 20, 2022.

(51) Int. Cl. A61B 5/02; A61B 5/00; A61B 5/0205; A61B 5/024; A61B 5/1455; G06F 9/44; H04L 67/12

(52) U.S. Cl. CPC A61B 5/02028; A61B 5/0205; A61B 5/7246; A61B 5/742; G06F 9/44; H04L 67/12; A61B 5/024; A61B 5/14551; G06F 2218/00

(57) ABSTRACT

A system for evaluating a cardiac pumping function includes an oximeter which is attached to a patient for the purpose of recording a pulse oximeter waveform. A computer is connected to the oximeter to receive metric information from the waveform. With this information, the computer determines the value and location of a second derivative acceleration, d²A/dt² in the waveform, which indicates the rate of rise/fall of the waveform. A comparator in the computer then compares this with the value and location of maximum second derivative acceleration, d²A/dt², in earlier waveforms. With this comparison, the computer identifies a trend which can be clinically used to evaluate the efficacy of a cardiac pumping function.

This divisional application claims priority to U.S. patent application Ser. No. 17/580,300, filed Jan. 20, 2022. The entire contents of application Ser. No. 17/580,300 are hereby incorporated by reference herein.

### FIELD OF THE INVENTION

The present invention pertains to systems and methods for monitoring and evaluating a cardiac pumping function. More particularly, the present invention pertains to systems and methods that evaluate cardiac pumping functions which are based on dynamic changes in blood pulse waveforms measured by an oximeter. The present invention is particularly, but not exclusively, useful for evaluating cardiac pumping functions by comparing the maximum second derivatives from a sequence of successive pulse oximeter waveforms, to assess the rise or fall of the waveforms as being indicative of the efficacy of the cardiac pumping function.

### BACKGROUND OF THE INVENTION

A pulse oximeter waveform is well known in the pertinent art as a graphical indication of the blood pressure response to a heart muscle function. Specifically, a pulse waveform shows the change in the amplitude A of blood pressure during a single contraction of the heart muscle. These waveforms are relatively short in duration and are, therefore, typically presented and considered as a continuous succession of pulse waveforms.

When considered individually each pulse waveform provides visual information of the velocity at which the amplitude A of the waveform is increasing or decreasing. Mathematically, this information is referred to as a first derivative, dA/dt. In addition to first derivative changes in velocity, pulses may also exhibit a rise or fall in amplitude of the entire waveform. This rise and/or fall of the waveform provides information about the acceleration of the waveform's amplitude A and is mathematically referred to as a second derivative, d²A/dt².

At the point of care, e.g., during surgery, information regarding changes in a heart muscle function can be quite helpful. Specifically, by monitoring a second derivative for the rise and/or fall of pulse waveforms, medical personnel can determine the beneficial or detrimental effect surgical activity may have had on heart muscle function. With this information, appropriate corrective action can be taken. In the event, it is obvious that corrective action, if needed, must be taken as soon as possible, i.e., immediately.

With the above in mind, it is an object of the present invention to provide a system and method for immediately evaluating a cardiac pumping function. Another object of the present invention is to provide a system and method for evaluating heart muscle function to determine when immediate corrective action may be advisable. Yet another object of the present invention is to provide a system and method for evaluating a cardiac pumping function by monitoring the acceleration of the pulse oximeter waveform as indicated by the second derivative of its amplitude, d²A/dt². Still another object of the present invention is to provide a system and method for monitoring a cardiac pumping function which is simple to use, is easy to manufacture, and is comparatively cost effective.

### SUMMARY OF THE INVENTION

A system and method for evaluating a cardiac pumping function requires attaching an oximeter to a patient to monitor a pulse oximeter waveform of the patient. A computer is then connected to the oximeter for receiving metric information from the pulse oximeter waveform. This metric information is received directly from the computer as input for calculating the rate of rise or fall of the pulse oximeter waveform per unit time. Mathematically, the rate of rise/fall of a pulse oximeter waveform is expressed as a second derivative of the waveform's amplitude A, d²A/dt². This second derivative is particularly important because it immediately provides an early detection, from a single pulse waveform, of an indication in trends for the overall heat muscle function.

A comparator, which is included with the computer, compares each pulse oximeter waveform with the immediately preceding waveform to calculate the second derivative d²A/dt². Furthermore, the computer identifies a maximum value for the second derivative and its location in the pulse oximeter waveform. This value and location information is then compared with similar value and location information obtained from earlier pulse oximeter waveforms to identify a trend with which to evaluate a cardiac pumping function.

In detail, each pulse oximeter waveform has a time interval that begins at a time tₒ and ends at a time tₑ. A plurality of time segments Δt can be identified between tₒ and tₑ with each time segment Δt having a respective amplitude A. There are two mathematical expressions of interest here for describing a change in A with respect to each time segment ΔA. The first expression is a velocity term which describes a change in the value of A as a function of time. Mathematically, this velocity term is a first derivative which is expressed as “dA/dt”. Stated differently, in the context of the present invention, the first derivative, dA/dt, describes the slope, i.e., shape, of the pulse oximeter waveform. The second expression of interest is an acceleration term that describes a change of the velocity term as a function of time. Mathematically this acceleration term is a second derivative which is expressed as, “d²A/dt²”. In the context of the present invention, the second derivative, d²A/dt², describes the rise and fall of the pulse oximeter waveform. As a practical consideration, it is the second derivative that is indicative of blood flow volume and thus, the efficacy of a cardiac pumping function.

For the present invention, the value and location of a maximum second derivative is determined for each consecutive pulse oximeter waveform. The value and location for the maximum second derivative of each pulse oximeter waveform is then compared with the value and location of the maximum second derivative in the immediately preceding waveform. The purpose here is to determine a trend in the value of successive second derivatives for a comparative evaluation that is helpful for determining the efficacy of a cardiac pumping function.

For the evaluation of a cardiac pumping function, a rise in the value of the second derivative is indicative of an improving function. On the other hand, a drop in the value of the second derivative is indicative of a worsening function. Most likely the maximum value of the second derivative for each pulse oximeter waveform occurs during a plurality of time segments Δt immediately following to. In any event, the present invention envisions the use of a visual display for showing trends in the maximum value of the second derivative, to thereby determine the efficacy of the cardiac pumping function.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of components of a system for evaluating a cardiac pumping function in accordance with the present invention;

FIG. 2 is a graph of a portion of a pulse oximeter waveform showing a mathematical first derivative expression for the velocity (i.e., slope) of the waveform;

FIG. 3A is a graph showing a mathematical second derivative expression for the acceleration (i.e., rise) of the waveform;

FIG. 3B is a graph showing a mathematical second derivative expression for the deceleration (i.e., fall) of the waveform; and

FIG. 4 is a composite graph showing the rise and fall of a pulse oximeter waveform resulting respectively from a positive second derivative (rise) and a negative second derivative (fall).

### DESCRIPTION OF THE PREFERRED EMBODIMENTS

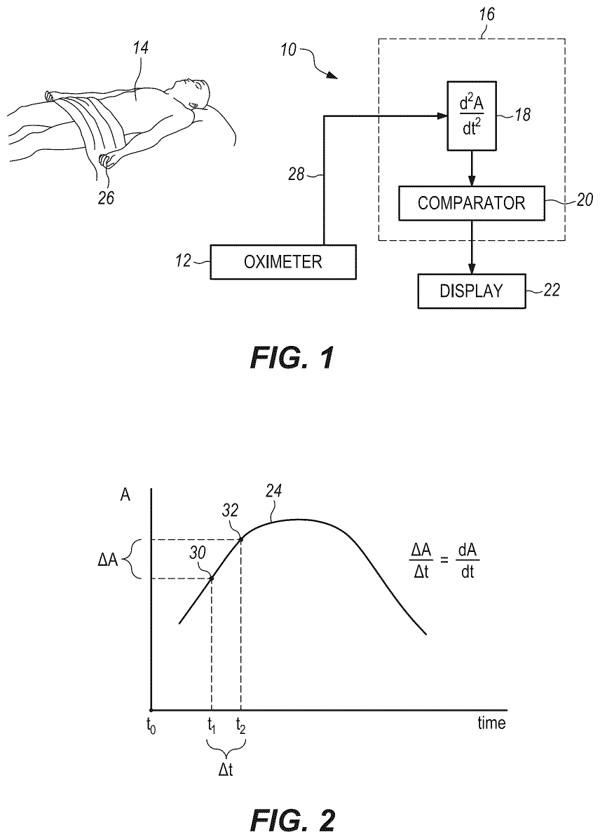

Referring initially to FIG. 1 a system for evaluating a cardiac pumping function is shown and is generally designated 10. As shown, the system 10 includes an oximeter 12 which can be connected with a patient 14 for the purpose of monitoring blood flow characteristics of the patient 14. FIG. 1 also shows that the system 10 includes a computer 16 which is attached to the oximeter 12, and that the computer 16 includes a differentiator 18 and a comparator 20. A display 22 is provided to present clinical results of measurements from the oximeter 12 that are pertinent to the blood flow characteristics of the patient 14. Specifically, these blood flow characteristics are based on measurements of a pulse oximeter waveform 24 (see FIG. 2) that is obtained by the oximeter 12.

Operationally, the oximeter 12 is typically connected with a finger 26 of the patient 14 to measure and record the physical characteristics of the patient's pulse oximeter waveform 24. The obtained measurements are then transmitted as metric information to the computer 16 via an electronic connection 28. Of particular interest for the present invention are mathematical expressions which are based on this metric information. Specifically, these mathematical expressions are first and second derivatives which are generated by the differentiator 18 in the computer 16. More specifically, the mathematical expressions are pertinent to changes in the pulse oximeter waveform 24.

FIG. 2 is a graphical presentation of an exemplary portion of a pulse oximeter waveform 24 whereon a change in the amplitude A of the pulse oximeter waveform 24 is shown as a function of time. Mathematically, such a change is expressed as ΔA/Δt (dA/dt). This expression is referred to as a first derivative, which establishes the “slope” of the waveform 24. The expression ΔA/Δt, or dA/dt, is also commonly often referred to as the “velocity” of the waveform 24.

Graphically, a change in the amplitude, ΔA, of the waveform 24 is shown in FIG. 2 to occur between points 30 and 32 during the time interval Δt between t1 and t2. In accordance with the present invention, this first derivative dA/dt, that occurs during the time interval t1 to t2, is used by the comparator 20 of computer 16 for comparison with the first derivative of the waveform 24 during the immediately subsequent same time interval Δt between t2 and t3. As disclosed below, this comparison is done to determine an acceleration of amplitude A of waveform 24.

Another mathematical expression of interest for the present invention is the second derivative of the pulse oximeter waveform 24, d²A/dt². This derivative expresses the time rate of change of the first derivative. It is also commonly referred to as the “acceleration” of the pulse oximeter waveform 24. This second derivative, i.e. acceleration, is of singular importance for the system 10 as it mathematically expresses the rise and/or fall of the waveform 24 as a function of time. Stated differently, as a practical consequence, the rise and fall of a waveform 24 is indicative of the volume of blood flow; with a rise being indicative of improved blood flow for the patient 14, and a fall (or drop) being indicative of a worsening of his/her blood flow condition.

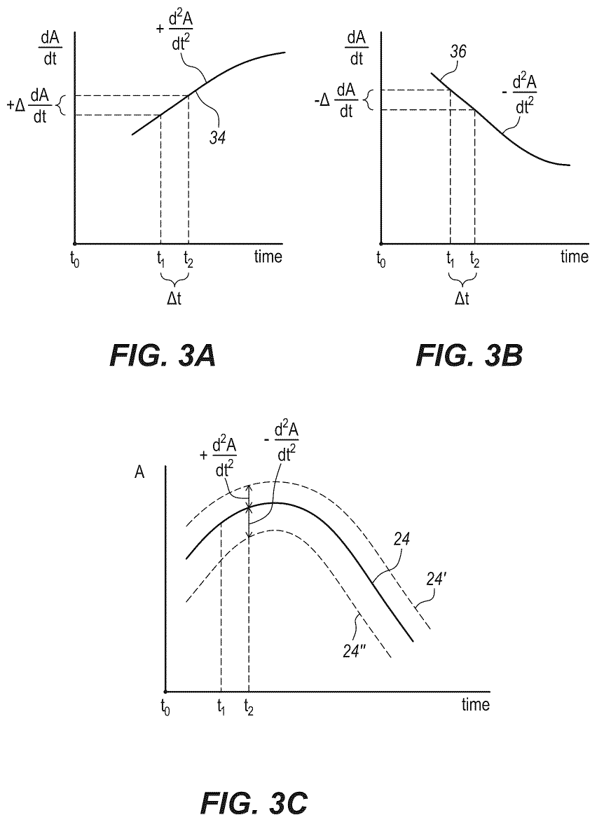

In FIG. 3A, the line curve 34 represents an increasing second derivative (+d²A/dt²), which indicates an acceleration in the magnitude of A. On the other hand, the line curve 36 in FIG. 3B represents a decreasing second derivative (−d²A/dt²), which indicates a deceleration in the magnitude of A. The consequences of these accelerations and decelerations are shown in FIG. 4.

The pulse oximeter waveform 24 shown in FIG. 4 is representative of a constant waveform 24 in which the amplitude A of the waveform 24 has neither accelerated nor decelerated. However, when the comparator 20 in computer 16 detects a second derivative change in the amplitude A, the display 22 presents a visual indication of the change. Specifically, as shown in FIG. 4 an acceleration will show on the display 22 as a movement of the waveform 24 toward a raised position shown for a waveform 24′. A deceleration, however, will show on the display 22 as a movement of the waveform 24 toward a lower position shown for a waveform 24″. As noted above, these movements provide valuable information to an attending physician at the time of care for an immediate response, if needed.

While the particular System and Method for Evaluating Cardiac Pumping Function as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for evaluating a cardiac pumping function which comprises the steps of:
attaching an oximeter to a patient to monitor a pulse oximeter waveform of the patient;
providing metric information received from the pulse oximeter waveform as input to a computer for calculating the rate of rise of the pulse oximeter waveform per unit time;
expressing the rate of rise of the pulse oximeter waveform mathematically as a second derivative; and
comparing a maximum value of the second derivative to a previously calculated value of the second derivative to determine the state of the cardiac pumping function.

2. The method of claim 1 wherein each pulse oximeter waveform has a time interval that begins at a time tₒ and ends at a time tₑ, with a plurality of time segments Δt therebetween, wherein each time segment Δt of the pulse oximeter waveform has a respective amplitude A, and wherein the mathematical expression for the second derivative is d²A/dt² and has a respective value for each time segment Δt.

3. The method of claim 2 further comprising the step of calculating the second derivative of A over the entire time interval from tₒ to tₑ at each time segment Δt.

4. The method of claim 3 further comprising the step of identifying a time segment Δt having a maximum value of the second derivative of A in the pulse oximeter waveform.

5. The method of claim 4 further comprising the step of comparing the location for the maximum value of the second derivative with the value of the second derivative at the same location in the immediately preceding waveform to determine a trend in the value of the second derivative.

6. The method of claim 5 wherein a rise in the second derivative is indicative of an improving cardiac pumping function and a drop in the second derivative is indicative of a worsening cardiac pumping function.

7. The method of claim 6 wherein the maximum value of the second derivative occurs during a plurality of time segments Δt immediately following tₒ.

8. A non-transitory, computer-readable medium having executable instructions stored thereon that direct a computer system to perform a process for evaluating a cardiac pumping function, the medium comprising instructions for:
receiving metric information from a pulse oximeter waveform as input to a computer for calculating the rate of rise of the pulse oximeter waveform per unit time;
expressing the rate of rise of the pulse oximeter waveform mathematically as a second derivative; and
comparing a maximum value of the second derivative to a previously calculated value of the second derivative to determine the state of the cardiac pumping function.

9. The medium of claim 8 wherein the pulse oximeter waveform has a time interval that begins at a time tₒ and ends at a time tₑ, with a plurality of time segments Δt therebetween, wherein each time segment Δt of the pulse oximeter waveform has a respective amplitude A, and wherein the mathematical expression for the second derivative is d²A/dt² and has a respective value for each time segment Δt.

10. The medium of claim 9 further comprising instructions for:
calculating the second derivative of A over the entire time interval from tₒ to tₑ at each time segment Δt; and
identifying a time segment Δt having a maximum value of the second derivative of A in the pulse oximeter waveform.

11. The medium of claim 10 further comprising an instruction for comparing the location for the maximum value of the second derivative with the value of the second derivative at the same location in the immediately preceding waveform to determine a trend in the value of the second derivative.

12. The medium of claim 11 further comprising an instruction for displaying a rising trend in the second derivative as indicative of an improving cardiac pumping function and a dropping trend in the second derivative as indicative of a worsening cardiac pumping function.

13. The medium of claim 12 wherein the maximum value of the second derivative occurs during a plurality of time segments Δt immediately following tₒ.

14. A method for measuring changes of an amplitude “A” in a blood flow waveform of a patient to determine a blood flow value which comprises the steps of.
Dividing the time interval of the blood flow waveform into a pluraliy of consecutive time segments Δt, wherein each time segment Δt of the waveform has a respective blood flow amplitude A and a respective location in the blood flow waveform;
Determine a change in blood amplitude Δt between adjacent time segment Δt of the blood flow waveform; and
Comparing the change of Δt between a downstream time segment Δt and its adjacent upstream time segment Δt at a redetermined location in the blood flow waveform to establish the blood flow value.

15. The method of claim 14 further comprising the step of using a pulse oximeter to present the blood flow waveform.

16. The method of claim 15 wherein the steps are performed using a computer.

17. The method of claim 16 wherein the change of Δt between adjacent time segments Δt is evidenced by a rise/fall rate of A at respective predetermined locations in the time segments, wherein the rise/fall rate is mathematically expressed as d²A/dt².

18. The method of claim 17 wherein the predetermined location on the waveform for determining blood flow value is where the magnitude of the second derivative d²A/dt² has a maximum value.

19. The method of claim 18 wherein changes in the second derivative d²A/dt² between successive time segments Δt provide an indication of trends in the patient's overall heart function.

20. The method of claim 19 wherein changes in the second derivative d²A/dt² between successive time segments Δt require immediate corrective action.
